(19) United States
(12) Patent Application Publication
(10) Pub. No.: US 2025/0067734 A1
(43) Pub. Date: Feb. 27, 2025

(54) Fluorescent Cellulose Particles

(71) Applicant: Asahi Kasei Kabushiki Kaisha, Tokyo (JP)

(72) Inventors: Maki Kato, Tokyo (JP); Hirokazu Nagai, Tokyo (JP); Yoshiyuki Sasajima, Tokyo (JP)

(73) Assignee: Asahi Kasei Kabushiki Kaisha, Tokyo (JP)

(21) Appl. No.: 18/711,135

(22) PCT Filed: Oct. 3, 2022
(86) PCT No.: PCT/JP2022/036982

§ 371 Date: May 17, 2024

(30) Foreign Application Priority Data
Nov. 18, 2021 (JP) 2021-187855

(51) Int. Cl. G01N 33/548; C09K 11/02; C09K 11/06

(52) U.S. Cl. CPC G01N 33/548; C09K 11/025; C09K 11/06; C09K 2211/182

(57) ABSTRACT

The present invention provides fluorescent cellulose microparticles which are capable of reducing deployment failure by improving the deployability during deployment in immunochromatography, while maintaining sufficient color developability and dispersion stability if used for immunochromatography. The present invention relates to: fluorescent cellulose particles, each of which contains a cellulose particle, a fluorescent dye compound and a heterocyclic compound represented by general formula (1) (wherein R₁ represents a functional group that has an affinity for a biological substance; and R₂ represents an ether bond part bonded to the cellulose particle), and which are characterized in that, per 1 g of the fluorescent cellulose particles, the content of the cellulose particles is 30% by mass to 90% by mass, the content of the fluorescent dye compound is 1% by mass to 40% by mass, and the content of the heterocyclic compound is 3% by mass to 50% by mass; and a diagnostic agent and an immunochromatography kit, each of which comprises the fluorescent cellulose particles.

### FIELD

The present invention relates to fluorescent cellulose particles, as well as a diagnostic agent and immunochromatography kit using the same.

### BACKGROUND

Conventionally, as one of the immunoassays that utilize a specific antigen-antibody reaction to detect a target substance consisting of a specific antigen or antibody, the agglutination method comprising specifically bonding, through an immunological reaction, a substance to be detected in a sample to an antibody or antigen supported on micro particles and measuring the state of aggregation of the micro particles produced by this bonding is commonly used because it is a simple measurement method and enables visual judgment. Furthermore, other immunoassays such as radioimmunoassay, enzyme immunoassay, and immunofluorescence assay are also widely used. A method for visual detection of a target substance using a substance that immunologically binds to the target substance according to a combination of an immune reaction and the chromatography principle is referred to as immunochromatography, and has been widely used in recent years.

Immunochromatography is the following measurement method: an antibody (or antigen) against an antigen (or antibody) as a substance to be detected is immobilized on a chromatographic medium to create a reaction site on a chromatographic medium as a stationary phase, and detection micro particles carrying an antibody (or antigen) capable of binding to the substance to be detected and a sample containing the substance to be detected, while brought into contact with each other—through this contact, the antibody (or antigen) on the particle for detecting antibody (or antigen) reacts with the antigen (or antibody) in the sample to create a complex consisting of the detection particle, the antibody (or antigen) on the particle, and the antigen (or antibody) in the sample— are moved on the chromatographic medium to bring the sample into contact with the reaction site. As a result, the complex is bonded to the immobilized antibody (or immobilized antigen) at the reaction site, and the detection micro particles are captured, whereby the presence of the substance to be detected in the sample can be determined by visually determining whether or not the detection micro particles are captured. A diagnostic agent kit which uses this principle is referred to as an immunochromatography kit.

In the immunochromatography kit and agglutination method described above, colored particles are often used as the detection micro particles to facilitate visual determination. As such detection micro particles, colloidal metal micro particles that develop a natural color depending on their particle size and preparation conditions, micro particles of colored latex micro particles made of synthetic polymers, and colored latex micro particles obtained by polymerizing monomers with a colorant are known. Furthermore, Patent Literature 1 below reports colored micro particles having high color development properties obtained from cellulose micro particles as a raw material. However, these micro particles have problems such as easy discoloration and limited color development properties, and further improvement in performance is desired. Thus, in recent years, fluorescent nanoparticles have attracted attention as new detection micro particles.

Fluorescent reagents used for detection and quantification of biomolecules using fluorescent nanoparticles have high color development properties and are used as highly sensitive reagents. For example, Patent Literature 2 below discloses fluorescent latex micro particles containing a fluorescent dye compound introduced into latex micro particles obtained by polymerizing styrene and acrylic acid. Furthermore, Patent Literature 3 below describes that fluorescent silica micro particles containing a fluorescent dye compound can be obtained by synthesizing a fluorescent dye compound, a silane coupling agent, and a silane compound.

However, these fluorescent nanoparticles have problems: for example, since an introduction amount of fluorescent dye compound is small, an immunochromatography kit does not provide satisfactory color development properties, and the particles tend to aggregate during storage, resulting in clogging and false positives when deployed with the immunochromatography kit.

Aiming to solve such problems, Patent Literature 4 below discloses fluorescent cellulose micro particles, and reports that when cellulose micro particles having a specific shape and a particle size within a specific range contain a fluorescent dye compound in a specific content range, fluorescent cellulose micro particles having high color development properties and suitable particle dispersion stability can be obtained, and additionally, high sensitivity of the immunochromatography kit can be achieved.

However, in Patent Literature 4 below, there is no sufficient study on the deployability of the particles when used in immunochromatography, and there is no mention of achieving both sensitivity/dispersion stability and deployability.

### CITATION LIST

### Patent Literature

[PTL 1] WO 2011/062157
[PTL 2] Japanese Patent No. 5317899
[PTL 3] Japanese Patent No. 5416039
[PTL 4] Japanese Patent No. 6148033
[PTL 5] Japanese Patent No. 3401170
[PTL 6] WO 2018/043687

### SUMMARY

### Technical Problem

In view of the problems of the prior art described above, the object of the present invention is to provide fluorescent cellulose micro particles which can improve deployability and reduce poor deployment during immunochromatography while maintaining sufficient color development properties and dispersion stability when used in immunochromatography. Further, by improving the deployability, background coloration during deployment is improved, and a suitable S/N ratio can be achieved even near the detection limit concentration.

### Solution to Problem

As a result of rigorous investigation and repeated experimentation to achieve the object described above, the present inventors have surprisingly discovered that when a fluorescent dye compound within a specific content range binds to cellulose particles and when a compound having a heterocyclic structure within a specific content range binds to cellulose particles, during use in immunochromatography, deployability during immunochromatography deployment is improved while sufficient color development intensity and particle dispersion stability are maintained, and additionally, the background coloration during deployment is improved and a suitable S/N ratio can be obtained even near the detection limit concentration, and based on this knowledge, have completed the present invention.

Specifically, the present invention is as described below.

[1] Fluorescent cellulose particles, comprising cellulose particles, a fluorescent dye compound, and a heterocyclic compound represented by general formula (1) below:

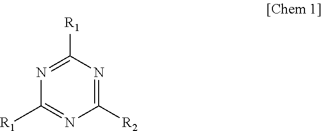

where R₁ is a functional group having affinity with a biological material, and R₂ is an ether bond to the cellulose particles, wherein per gram of the fluorescent cellulose particles, a content of the cellulose particles is 30% by mass or more and 90% by mass or less, a content of the fluorescent dye compound is 1% by mass or more and 40% by mass or less, and a content of the heterocyclic compound is 3% by mass or more and 50% by mass or less.
   [2] The fluorescent cellulose particles according to [1], wherein R₁ of the heterocyclic compound is Cl and/or OH.
   [3] The fluorescent cellulose particles according to [1] or [2], wherein an average particle diameter of the fluorescent cellulose particles is 9 nm or more and 500 nm or less.
   [4] The fluorescent cellulose particles according to any one of [1] to [3], wherein the fluorescent dye compound is bonded to an OH group of the cellulose particles, and the heterocyclic compound is bonded to an OH group of the cellulose particles.
   [5] The fluorescent cellulose particles according to any one of [1] to [4], wherein the fluorescent dye compound is a europium complex.
   [6] The fluorescent cellulose particles according to any one of [1] to [5], wherein the biological material is supported via physical adsorption.
   [7] The fluorescent cellulose particles according to [6], wherein the biological material is a protein, a peptide, or a nucleic acid.
   [8] The fluorescent cellulose particles according to [7], wherein the protein is an antigen or an antibody.
   [9] A diagnostic agent comprising the fluorescent cellulose particles according to any one of [1] to [8].
   [10] An immunochromatography kit comprising the fluorescent cellulose particles according to any one of [1] to [8].

### Advantageous Effects of Invention

When used as color developing particles in immunochromatography, the fluorescent cellulose particles of the present invention exhibit excellent deployability while maintaining color development properties and dispersion stability. Furthermore, by improving poor deployment and background coloration, a suitable S/N ratio can be achieved even near the detection limit concentration.

### BRIEF DESCRIPTION OF DRAWINGS

FIG. 1 is a schematic diagram of an immunochromatography (test) strip used as an evaluation standard for deployability in a method for determining color development. Regarding each of the coloration 4 mm upstream of the membrane and the coloration of the absorbent pad, the case in which no coloration was observed is evaluated as (−), the case in which coloration was observed is evaluated as (+), and case in which strong coloration was observed is evaluated as (++).

### DESCRIPTION OF EMBODIMENTS

The present invention will be described in detail below.

A first embodiment of the present invention is directed to fluorescent cellulose particles, comprising cellulose particles, a fluorescent dye compound, and a heterocyclic compound represented by general formula (1) below:

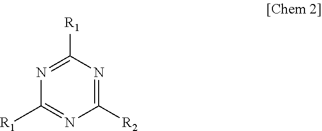

where R₁ is a functional group having affinity with a biological material, and R₂ is an ether bond to the cellulose particles, wherein per gram of the fluorescent cellulose particles, a content of the cellulose particles is 30% by mass or more and 90% by mass or less, a content of the fluorescent dye compound is 1% by mass or more and 40% by mass or less, and a content of the heterocyclic compound is 3% by mass or more and 50% by mass or less.

The content of the cellulose particles in the fluorescent cellulose of the present embodiment is 30% by mass or more and 90% by mass or less, preferably 35% by mass or more and 85% or less per gram of the fluorescent cellulose particles.

In general formula (1), though R₁ is preferably a functional group having affinity with a biological material and R₂ is an ether bond to the cellulose particles, R₁ may be an ether bond to the cellulose particles and R₂ may be a functional group having affinity with a biological material.

The particle size of raw material cellulose particles used for producing the fluorescent cellulose particles of the present embodiment is smaller than the particle size of the ultimately obtained fluorescent cellulose particles, and specifically, is preferably 3 nm or more and 480 nm or less, and more preferably 6 nm or more and 460 nm or less, considering that the particle size increases due to modification with the fluorescent dye compound/heterocyclic compound.

The raw material of the fluorescent cellulose particles of the present embodiment may be any cellulose, and the cellulose source is not particularly limited. When a raw material other than cellulose is used, a sufficient amount of the fluorescent dye compound cannot be introduced due to the problem of chemical reactivity when introducing the fluorescent dye compound. For example, Patent Literature 5 describes that if latex particles contain a colorant in an amount exceeding 12% by mass, it is highly likely that the pores of the immunochromatography kit will become clogged. Furthermore, though Patent Literature 6 discloses fluorescent particles containing 10% by mass or more of a cohesive luminescent material for a diagnostic agent, usable luminescent materials are limited. It is extremely difficult in the first place to introduce a large amount of dye or fluorescent chemical substance into latex, and even if a large amount thereof could be introduced, the surface structure may collapse and the sphericity may become extremely poor, and thus, latex is not preferable for introducing a large amount of dye or chemical substance. Conversely, cellulose can maintain its structure even when it contains a large amount of dye or chemical substance. Thus, high reactivity and high content can be achieved precisely because cellulose has an abundance of hydroxyl groups. Therefore, cellulose is suitable as the material for the detection particles for immunochromatography. Further, the content of the fluorescent dye compound can be calculated from the weight change before and after the fluorescent dye compound treatment. The proportion of the fluorescent dye compound component is calculated using the weight of the particles recovered after the treatment and the weight of the cellulose particles before the treatment after being completely dried.

When the weight of the cellulose particles before treatment is unknown, the fluorescent cellulose particles are subjected to a cellulase treatment, an acid treatment, or a base treatment to reduce the degree of polymerization. Thereafter, the sample is dissolved in heavy water, measured by ¹³C-NMR using FT-NMR, and the degree of substitution is calculated. The content of the fluorescent dye compound may be calculated from the degree of substitution. At that time, the cellulase, acid, and base to be used are not particularly limited, and examples of the cellulase include Onozuka RS (manufactured by Yakult Pharmaceutical Co., Ltd.), Cellsoft (manufactured by Novo Nordix), and Meicelase (manufactured by Meiji Seika Co., Ltd.), examples of the acid include hydrochloric acid, sulfuric acid, and nitric acid, and examples of the base include alkali.

Furthermore, when the weight of the cellulose particles before treatment is unknown and the fluorescent dye compound contains a nitrogen atom, the nitrogen element content may be measured by a nitrogen quantitative device CHN coder using an optical emission spectrometry method, and the content of the fluorescent dye compound may be calculated from the measured nitrogen element content.

The type of the fluorescent dye compound is not particularly limited, and examples thereof include fluoresceins having an active substituent such as an N-hydroxysuccinimide ester group, ester group, carboxyl group, maleimide group, isocyanate group, isothiocyanate group, cyano group, halogen group, aldehyde group, paranitrophenyl group, diethoxymethyl group, or epoxy group, rhodamines, coumarins, cyanines, and other compounds emitting fluorescence and rare earth complexes containing europium. Specific examples of the fluorescent dye compound include fluorene, fluorene-9-acetic acid, fluorene-2-carboxaldehyde, 9-fluorene-1-carboxylic acid, 9-fluorene-4-carboxylic acid, 9-fluorene oxime, 9-fluorenemethylsuccinimidyl carbonate, 9-fluoretriphenylphosphonium bromide, 5-aminofluorescein, disodium 8-amino-1,3,6-naphthalene trisulfonate hydrate, sulforodamine B, ethidium bromide, 6-aminofluorescein, rhodamine B, Rhodamine 6G, ammonium 8-anilino-1-naphthalenesulfonate, sodium 8-anilino-1-naphthalenesulfonate, magnesium 8-anilino-1-naphthalenesulfonate, 2,3-naphthalenedialdehyde, sodium calcein, calcein, coumarin 102, coumarin 314, coumarin 343, AMCA, 5-carboxyfluorescein hydrate, 6-carboxyfluorescein hydrate, fluorescein chloride, 2′,7′-dichlorofluorescein, 2′,7′-dichlorofluorescein sodium, 2,3-diaminonaphthalene, dimidium bromide, 2,3-diphenylmale K, fluorescein, uranine, fluorescein diacetate, coumarin-3-carboxylic acid, 7-hydroxycoumarin-3-carboxylic acid, 4-dimethylaminoazobenzene-4′-carboxylic acid, 7-methoxycoumarin-3-carboxylic acid, pinacyanol chloride, pinacyanol iodide, pyranine, N-(1-pyrenyl)maleimide, rhodamine 6G, rhodamine B, sulfonefluorescein, N-succinimidyl 7-methoxycoumarin-3-carboxylate, potassium tetrabromofluorescein, acid red 87, 2′,4′,5′,7′-tetrabromo-3,4,5,6-tetrachlorofluorescein, 9H-fluoren-2-yl isocyanate, fluorescein 5-isothiocyanate, acid red 92, 3,4,5,6-tetrachlorofluorescein, tetraiodofluorescein, 5-(4,6 dichlorotriazinyl)aminofluorescein (DTAF), erythrosin B, 5-(6-)carboxytetramethylrhodamine-NHS ester, DYLIGHT-405-NHS ester, DY550-NHS ester, DY630-NHS ester, DY-631, DY-633, DY-635, DY-636, DY-650, DY-651-NHS ester, DY-777-NHS ester (Dy- are manufactured by Dyomics), sodium[4′-(4′-amino-4-biphenylyl)-2,2′:6′,2″-terpyridine-6,6″-diylbis(methyliminodiacetate)] europiumate (III) (ATBTA-Eu3+), BODYIPY650/665, ROX, TAMRA, CFSE, Cyto350, Cyto405, Cyto415, Cyto488, Cyto500LSS, Cyto505, Cyto510SS, Cyto514LSS, Cyto520LSS, Cyto532, Cyto546S, Cyto555, Cyto590, Cyto610, Cyto610, Cyto633, Cyto647, Cyto670, Cyto680, Cyto700, Cyto750, Cyto770, Cyto780, Cyto800 (Cyto- are manufactured by Cytodiagnostics), ATTO532, ATTORho6G, ATTO542, ATTO550, ATTO565, ATTORho3B, ATTORho11, ATTORho12, ATTOThio12, ATTORho101, ATTO590, ATTORho13, ATO594, ATO610, ATO620, ATORho14, ATO633, ATO647N, ATO647, ATO655, ATOOxa12, ATTO665, ATTO680, ATTO700, ATTO725, and ATTO740 (ATTO- are manufactured by ATTO-TEC). The fluorescence wavelength of each of these fluorescent dye compounds is preferably in the range of 400 nm or more, which does not overlap with the wavelength of water or protein during detection. The upper limit of the wavelength is not particularly limited, and the higher the wavelength, the better. The fluorescent dye compound more preferably has a wavelength of 500 nm or more. The fluorescent dye compound is more preferably a europium complex.

For chemical bonding between the fluorescent cellulose particles and the fluorescent dye compound, there are a method in which the hydroxyl groups of cellulose are directly linked to the fluorescent dye compound, and a method in which the hydroxyl groups of cellulose are linked via some sort of compound as a spacer. When including a large amount of fluorescent dye compound, there is a limit to direct linking alone, but via a spacer, it becomes possible to introduce a large amount. When linking using a spacer, the type of spacer is not particularly limited, and examples thereof include cyanuric chloride, epichlorohydrin, 2-chloroethanamine, 11-chloroundecanethiol, formalin, silane coupling agents, epoxy-modified silicone crosslinking agents, glyoxal resins, and other compounds each having two or more moieties that react with hydroxyl groups.

The content of the fluorescent dye compound in the fluorescent cellulose particles of the present embodiment is 1% by mass or more and 40% by mass or less per gram of the fluorescent cellulose particles. When the content is less than 1% by mass, sufficient color development properties cannot be obtained as detection particles for an immunochromatography kit. Conversely, by setting the content to 40% by mass or less, concentration quenching originating from the fluorescent dye is suppressed, whereby fluorescence intensity is suitable, and sensitivity is excellent as an immunochromatography kit. A preferable lower limit is 5% by mass, and a preferable upper limit is 35% by mass.

The heterocyclic compound contained in the fluorescent cellulose particles of the present embodiment is a heterocyclic compound represented by the following general formula (1):

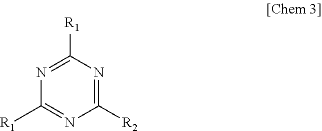

where R₁ is a functional group having affinity with a biological material, and R₂ is an ether bond to the cellulose particles.
R₁ is not particularly limited as long as it is a functional group having affinity with a biological material, and examples thereof include a halogen group, amino group, carboxyl group, thiol group, hydroxyl group, ether group, ester group, imine group, phenyl group, benzyl group, and aryl group. When actually introducing into cellulose, it is preferable to use, for example, cyanuric chloride, thiocyanuric acid, 2,4-bis(benzyloxy)-6-chloro-1,3,5-triazine, 2,4,6-triamino-1,3,5-triazine, 2-chloro-4,6-diamino-1,3,5-triazine, 2-chloro-4,6-diphenyl-1,3,5-triazine, 2-bromo-4,6-diphenyl-1,3,5-triazine, 2,4-dichloro-6-morpholino-1,3,5-triazine, 2-Chloro-4,6-dimethoxy-1,3,5-triazine, or 4-(4,6-dimethoxy-1,3,5-triazin-2-yl)-4-methylmorpholinium chloride. Cyanuric chloride is more preferable. R₂ is an ether bond to the cellulose particles. This can be a location of a single bond that forms an —O— bond (O is derived from cellulose) with an OH group of the cellulose. In the present description, in the case of a single bond, the content (%) of the heterocyclic compound is calculated based on the structure of general formula (1) excluding R₂.

The content of the heterocyclic compound in the fluorescent cellulose particles of the present embodiment is 3% by mass or more and 50% by mass or less per gram of fluorescent cellulose particles. By setting the content to 3% by mass or more, hydrophobic interaction between the particles and the cellulose deployment membrane used in the immunochromatography kit can be suppressed, whereby the fluidity of the particles in the deployment membrane is improved, and sufficient color development properties as detection particles for an immunochromatography kit is obtained. Conversely, by setting the content to 50% by mass or less, the particles do not aggregate due to hydrophobic interaction, whereby clogging and false positives do not occur during deployment. Thus, sufficient sensitivity as an immunochromatography kit can be obtained. A preferable lower limit of the content of the heterocyclic compound is 5% or more, and a preferable upper limit is 45% or less.

When the weight of the cellulose particles before treatment is unknown, the fluorescent cellulose particles are subjected to a cellulase treatment, an acid treatment, or a base treatment to reduce the degree of polymerization. Thereafter, the sample is dissolved in heavy water, measured by ¹³C-NMR using FT-NMR, and the degree of substitution is calculated. The contents of the fluorescent dye compound and the heterocyclic compound may be calculated from the degree of substitution. At that time, the cellulase, acid, and base to be used are not particularly limited, and examples of the cellulase include Onozuka RS (manufactured by Yakult Pharmaceutical Co., Ltd.), Cellsoft (manufactured by Novo Nordix), and Meicelase (manufactured by Meiji Seika Co., Ltd.), examples of the acid include hydrochloric acid, sulfuric acid, and nitric acid, and examples of the base include alkali. Furthermore, when the weight of the cellulose particles before treatment is unknown and the fluorescent dye compound contains a nitrogen atom, the nitrogen element content can be measured using a nitrogen quantitative device CHN coder using an optical emission spectrometry method, and the content of the fluorescent dye compound and heterocyclic compound may be calculated from the nitrogen element content.

The particle size of fluorescent cellulose particles or raw material cellulose particles of the present embodiment refers to the particle size obtained by measuring a cellulose particle dispersion of the cellulose particles dispersed in a liquid using a particle size distribution measuring device. “Average particle size” refers to the volume average median diameter of the measured values. Though there are particle size distribution measuring devices which apply various measurement principles, in the present embodiment, a particle size distribution measuring device using the dynamic light scattering method is used. As will be described later, in the Examples, a “Nanotrack particle size distribution analyzer UPA-EX150” manufactured by Nikkiso Co., Ltd., was used.

The average particle size of the fluorescent cellulose particles of the present embodiment is 9 nm or more and 500 nm or less. When the average particle size is within this range, aggregation is unlikely to occur during long-term storage, which is suitable for immunochromatography kits. When used as a diagnostic agent, the average particle size is preferably 20 nm or more and 500 nm or less. When it is 20 nm or more and 500 nm or less, it is possible to achieve both dispersion stability without agglomeration and deployability without clogging. However, to improve the sensitivity of the immunochromatography kit, two or more fluorescent cellulose particles which have different average particle sizes may be used in combination.

The fluorescent cellulose particles of the present embodiment can be used by supporting a biological material via physical adsorption. Examples of the physical adsorption include, but are not limited to, ionic bonds, coordinate bonds, metal bonds, hydrogen bonds, hydrophilic bonds, hydrophobic bonds, and van der Waals bonds. By supporting a biological material on the fluorescent cellulose particles using the various forces described above, it is possible to prepare particles having a function that fluorescent cellulose particles do not have.

The “biological material” supported on the fluorescent cellulose particles of the present embodiment refers to various substances obtained from living organisms, and the type thereof is not particularly limited. Examples thereof include collagen, gelatin, fibroin, heparin, hyaluronic acid, starch, chitin, chitosan, amino acids, peptides, proteins, nucleic acids, carbohydrates, fatty acids, terpenoids, carotenoids, tetrapyrroles, cofactors, steroids, flavonoids, alkanoids, polyketides, glycosides, enzymes, antibodies, antigens, carboxymethylcellulose, carboxyethylcellulose, and methylcellulose. By supporting these on the fluorescent cellulose particles, it becomes possible to improve the biocompatibility of the fluorescent cellulose particles and to use them in various bioassays and as diagnostic agents.

In the present embodiment, a substance which specifically binds to the substance to be tested is supported on the fluorescent cellulose particles, whereby the fluorescent cellulose particles can be used as a diagnostic agent. The substance to be tested refers to an object to be measured in a test such as an immune serum test, a blood test, a cell test, or a genetic test, and the type thereof is not particularly limited. Examples include cancer markers, hormones, infectious diseases, autoimmunity, plasma proteins, TDM, coagulation/fibrinolysis, amino acids, peptides, proteins, genes, and cells. More specific examples include CEA, AFP, ferritin, β2 micro, PSA, CA19-9, CA125, BFP, elastase 1, pepsinogen 1, 2, fecal occult blood, urinary β2 micro, PIVKA-2, urinary BTA, insulin, E3, HCG, HPL, LH, HCV antigens, HBs antigens, HBs antibodies, HBc antibodies, HBe antigens, HBe antibodies, HTLV-1 antibodies, HIV antibodies, Toxoplasma antibodies, syphilis, ASO, influenza A antigens, influenza A antibodies, influenza B antigens, influenza B antibodies, rota antigens, adenovirus antigens, rota adenovirus antigens, group A streptococcus, group B streptococcus, candida antigens, CD bacteria, cryptotrocus antigens, Vibrio cholerae, Neisseria meningitidis antigens, Granulobacillus elastase, Helicobacter pylori antibodies, O157 antibodies, O157 antigens, Leptospira antibodies, Aspergillus antigens, MRSA, RF, total IgE, LE test, CRP, IgG, A, M, IgD, transferrin, urinary albumin, urinary transferrin, myoglobin, C3/C4, SAA, LP(a), α1-AC, α1-M, haptoglobin, microtransferrin, APR score, FDP, D-dimer, plasminogen, AT3, α2PI, PIC, PAI-1, protein C, coagulation factor X3, type IV collagen, hyaluronic acid, GHbA1c, various antigens, various antibodies, various viruses, various bacteria, various amino acids, various peptides, various proteins, various DNA, and various cells.

Though the fluorescent cellulose particles can be dispersed in various solutions when the fluorescent cellulose particles of the present embodiment are used as a diagnostic agent, preferably, a dispersion thereof dispersed in a buffer solution having a pH of 5.0 or more and 11.0 or less is preferred. As a solution for dispersing the fluorescent cellulose particles, pure water or an organic solvent can be used. Examples thereof include a phosphate buffer, glycine buffer, Tris buffer, borate buffer, citrate buffer, MES buffer, methanol, ethanol, acetone, and tetrahydrofuran. The concentration of the buffer solution is not particularly limited, and buffer solutions of various concentrations commonly used as buffer solutions can be used. Furthermore, the concentration of fluorescent cellulose particles in the dispersion is not particularly limited and can be appropriately adjusted and used depending on the type, property, and concentration of the substance to be tested. The concentration of the fluorescent cellulose particles in the dispersion is preferably 0.001% by mass or more, and more preferably 0.002% by mass or more, because if the concentration is excessively low, detectability will be poor and high sensitivity cannot be achieved. Conversely, when the concentration is excessively high, poor deployment occurs due to concentration quenching or aggregation, and high sensitivity cannot be expected, and thus, the concentration is preferably about 10% by mass or less, and more preferably 1.0% by mass or less.

When the fluorescent cellulose particles of the present embodiment are used as a diagnostic agent, various sensitizers may be used to improve measurement sensitivity and promote antigen-antibody reactions. Furthermore, a blocking agent or the like may be used to suppress non-specific adsorption caused by other substances present in the sample. Though the fluorescent cellulose particles of the present embodiment can be used by being dispersed in an arbitrary liquid such as a diagnostic agent, they can also be used by being dispersed in another arbitrary solid, or by immobilizing the particles on a solid surface. Furthermore, by coloring the fluorescent cellulose particles, it is also possible to improve the visibility of the particles and the detection sensitivity.

The method for producing the cellulose particles included in the fluorescent cellulose particles of the present embodiment is not particularly limited. Though particles having a desired average particle size may be obtained by classification using a mechanical method such as wet grinding, in the present embodiment, the cellulose particles are prepared by dissolving cellulose in a good solvent therefor and using a coagulation liquid containing a mixture of water, an organic solvent, and ammonia. By using this method, it becomes possible to adjust the particle size of the cellulose particles obtained by adjusting the composition of the coagulation liquid. Though not intended to limit the production method of the cellulose particle material included in the fluorescent cellulose particles of the present embodiment, production methods 1 and 2 will be exemplified below.

### [Production Method 1: Preparation of Cellulose Particles]

Cellulose linter is dissolved in a good solvent for cellulose. A copper ammonia solution prepared by a known method is used as the good solvent. As the coagulation liquid, a mixed system of an organic solvent, water, and ammonia is primarily used. While stirring this coagulation liquid, the prepared cupric ammonia cellulose solution is added for coagulation. By further adding sulfuric acid for neutralization and regeneration, a slurry containing the cellulose particles of interest can be obtained. At this time, since the slurry is acidic due to the residual acid used for regeneration and contains impurities such as ammonium salts generated during neutralization, it is necessary to refine the slurry into a cellulose dispersion consisting of cellulose particles and a medium. As this refinement operation, repeated processes of centrifugation, decantation, and dilution with a dispersion medium liquid are used. The type of the dispersion medium liquid used at this time is not particularly limited, and the various hydrophilic solvents described above can be used in accordance with the purpose. Since the cellulose particles in the obtained cellulose particle dispersion sometimes aggregate during the refinement operation, in this case, a dispersion treatment using shearing, or the like can be performed. A high-pressure homogenizer is used as the means for applying shearing. The average particle size and CV of the cellulose particle dispersion element obtained in this manner are measured using a particle size distribution measuring device. CV is an abbreviation for Coefficient of Variation, which represents the degree of polydispersity in the particle size distribution of a cellulose particle dispersion on a volume basis, and is defined by the following formula (1). The smaller this value, the sharper the particle size distribution, which means that the sizes of the cellulose particles are more uniform, and the unit is (%).

$$CV ( \% ) = ( standard deviation in volume particle size distribution determined by particle size distribution measuring device ) / ( volume average median diameter determined by particle size distribution measuring device ) \times 100 \qquad formula ( l )$$

The obtained cellulose particle dispersion element can be used by adding a surfactant as needed. The cellulose particle dispersion can be used as-is in a never-dried state, and if necessary, can be prepared into cellulose particles by drying. The obtained cellulose particles are observed using an electron microscope, and the sphericity and aggregation constant are measured from the images thereof. Furthermore, the cellulose particles are dissolved in a cadoxene solution, and the average degree of polymerization is measured from the viscosity thereof. The average degree of polymerization of cellulose particles suitable for producing the fluorescent cellulose particles is 30 or more and 700 or less. When the average degree of polymerization is 30 or more and 700 or less, the uniformity of the particles can be maintained and the fluorescent dye compound can also be stably contained, resulting in stable quality when used in an immunochromatography kit. Thus, regarding the fluorescent cellulose particles, by controlling the degree of polymerization of the cellulose particles before dyeing and the average particle size within the ranges of the present invention, it is possible to produce fluorescent cellulose particles suitable for an immunochromatography kit. In order to produce the fluorescent cellulose particles, the lower limit of the average degree of polymerization of the cellulose particles is preferably 35 or more, and more preferably 40 or more. Furthermore, a preferable upper limit is 650, and more preferably 600.

### [Production Method 2: Preparation of Fluorescent Cellulose Particles]

The cellulose particles produced by the above production method 1 are added to an solvent and dispersed. These cellulose particles may be colored. Furthermore, examples of the organic solvent include methanol, ethanol, isopropyl alcohol, butanol, pentanol, hexanol, diethyl ether, isopropyl ether, dichloromethane, chloroform, carbon tetrachloride, ethyl acetate, methyl acetate, methyl ethyl ketone, cyclohexane, cyclopentane, tetrahydrofuran, toluene, hexane, water, and caustic soda, one or a mixture of two or more thereof may be used in accordance with the type of fluorescent dye compound. Furthermore, the cellulose particles which are the raw material of the fluorescent cellulose particles are of a cellulose II crystal type and thus have a low degree of crystallinity, the amount of fluorescent dye introduced into the cellulose particles can be significantly increased more than conventional latex particles or silica particles. In order to increase the amount of fluorescent dye introduced, the cellulose may be physically or chemically modified to introduce amino groups or thiol groups, and then reacted with the fluorescent dye compound.

After adding the fluorescent dye compound to a solution containing the cellulose particles, as appropriate, additives are added, the pH is adjusted, or heating or cooling is performed. The slurry contains unreacted substances and by-products of the fluorescent dye compound used in the reaction, and it is necessary to refine the fluorescent cellulose particles and the medium. As this refinement operation, repeated processes of centrifugation, decantation, and dilution with a dispersion medium liquid are used. The type of the dispersion medium liquid used at this time is not particularly limited, and the various hydrophilic or lipophilic solvents or solutions described above can be used in accordance with purpose.

Furthermore, after adding the heterocyclic compound, which is not the fluorescent dye compound, to the solution containing the fluorescent cellulose particles, as appropriate, additives are added, the pH is adjusted, or heating or cooling is performed. The slurry contains unreacted substances and by-products of the heterocyclic compound used in the reaction, and it is necessary to refine the fluorescent cellulose particles and the medium. The refinement operation is as described above.

The fluorescent cellulose particles of the present embodiment can be produced through the steps described above.

### EXAMPLES

The present invention will be specifically described below with reference to Examples and Comparative Examples, but the present invention is not limited to the Examples in any way. The primary measurement values of the Examples and Comparative Examples were obtained by the following methods.

### <Content of Fluorescent Dye Compound>

The proportion of the fluorescent dye compound component to the fluorescent cellulose particles can be calculated from the weight change before and after the fluorescent dye compound treatment. The proportion of the fluorescent dye compound component is calculated from the following formula (2) using the weight of the particles recovered after treatment and the weight of cellulose particles before treatment after absolute drying:

$$Fluorescent dye compound content ( \% ) = 1 - \{ ( weight of cellulose particles before treatment ) / ( weight of fluorescent cellulose particles after treatment with fluorescent dye compound ) \} \times 100 \qquad formula ( 2 )$$

### (When Weight of Cellulose Particles Before Treatment is Unknown)

After the fluorescent cellulose particles are subjected to a cellulase treatment, an acid treatment, or a base treatment, the sample is dissolved in heavy water to prepare a 3 to 5% by mass heavy water solution, which is measured by ¹³C-NMR (Avance 400 MHz) using FT-NMR, and the degree of substitution is calculated. The degree of substitution is calculated from the peak area of the fluorescent dye compound based on the C1 peak area of the cellulose. The content of the fluorescent dye compound is calculated from the degree of substitution and the molecular weight of the fluorescent dye compound.

### (When Weight of Cellulose Particles Before Treatment is Unknown and Fluorescent Dye Compound Contains Nitrogen Atom)

The nitrogen element content is measured by optical emission spectrometry using a nitrogen quantitative device CHN coder (manufactured by Yanako Analytical Industry Co., Ltd.) under the following measurement conditions. The content of the contained fluorescent dye compound is calculated from the measured nitrogen element content.

### Measurement Method: Self-Integration Method

Carrier gas: helium
Auxiliary gas: high purity oxygen
Auxiliary combustion method: helium-oxygen mixture method

### <Content of Heterocyclic Compound>

The proportion of the heterocyclic compound component to the fluorescent cellulose particles can be calculated from the weight change before and after the fluorescent cellulose particle treatment. The proportion of the heterocyclic compound component is calculated the following formula (3) using the weight of the particles recovered after treatment and the weight of the cellulose particles before treatment after absolute drying:

$$Heterocyclic compound content ( \% ) = 1 - \{ ( weight of fluorescent cellulose particles before treatment ) / ( weight of fluorescent cellulose particles after heterocyclic compound treatment ) \} \times 100 \qquad formula ( 3 )$$

### (When Weight of Cellulose Particles Before Treatment is Unknown)

The fluorescent cellulose particles having undergone the heterocyclic compound treatment are subjected to a cellulase treatment, an acid treatment, or a base treatment, and thereafter the sample is dissolved in heavy water to prepare a 3 to 5% by mass heavy water solution, which is measured by ¹³C-NMR (Avance 400 MHz) using FT-NMR, and the degree of substitution is calculated. The degree of substitution is calculated from the peak area of the heterocyclic compound based on the peak area of C1 of cellulose. The content of the heterocyclic compound is calculated from the degree of substitution and the molecular weight of the heterocyclic compound.

### (When Weight of Cellulose Particles Before Treatment is Unknown and Fluorescent Dye Compound Contains Nitrogen Atom)

The nitrogen element content is measured by optical emission spectrometry using a nitrogen quantitative device CHN coder (manufactured by Yanako Analytical Industries) under the following measurement conditions. The content of the contained heterocyclic compound is calculated from the measured nitrogen element content. When the fluorescent dye compound before the heterocyclic compound treatment also contains a nitrogen atom, the amount can be calculated based on the relative amount.

### Measurement Method: Self-Integration Method

Carrier gas: helium
Auxiliary gas: high purity oxygen
Auxiliary combustion method: helium-oxygen mixture method

### <Particle Size Measurement Method>

A slurry containing the cellulose particles is diluted with distilled water so that the cellulose particles are 0.005% by mass, and the slurry is used for measurement. Measurement is carried out using a “Nanotrack Particle Size Distribution Analyzer UPA-EX150” manufactured by Nikkiso Co., Ltd., which performs measurements using the dynamic light scattering method.

### <Method for Determining Sensitivity of Immunochromatography Evaluation>

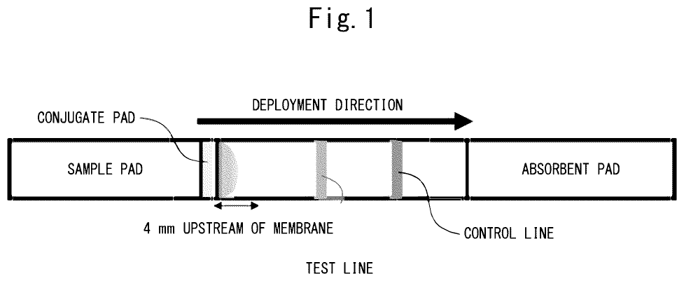

In the method for determining color development, color development intensity is evaluated using a fluorescent immunochromatography reader “DxCELL series HRDR-300” manufactured by Cellmic, LLC. Furthermore, in Table 1 below, as an evaluation standard for deployability, when the immunochromatographic strip after deployment was exposed to a UV lamp, the case in which no coloration was observed in each of 4 mm upstream of membrane and the absorbent pad shown in FIG. 1 is evaluated as (−), the case in which coloration was observed therein is evaluated as (+), and the case in which strong coloration was observed therein is evaluated as (++).

### Example 1

A copper ammonia cellulose solution having a cellulose concentration of 0.37% by mass, a copper concentration of 0.13% by mass, and an ammonia concentration of 1.00% by mass was prepared. A coagulation liquid having a tetrahydrofuran concentration of 87.5% by mass and a water concentration of 12.5% by mass was prepared. While slowly stirring 5000 g of the coagulation liquid using a magnetic stirrer, 500 g of the prepared copper ammonia cellulose solution was added thereto. After continuing stirring for approximately 5 seconds, 1000 g of 10% by mass sulfuric acid was added to perform neutralization and regeneration to obtain 6500 g of a slurry containing cellulose particles.

The obtained slurry was centrifuged at a speed of 10,000 rpm for 10 minutes. The precipitate was removed by decantation, ultrapure water was poured thereinto followed by stirring, and centrifugation was performed again. This operation was repeated several times until the pH reached 6.0 to 7.0, and thereafter, a dispersion treatment was performed using a high-pressure homogenizer to obtain 150 g of a cellulose particle dispersion. The average particle size of the obtained cellulose particles was measured and found to be 205 nm.

200 mg of sodium [4′-(4′-amino-4-biphenylyl)-2,2′:6′,2″-terpyridine-6,6″-diylbis(methyliminodiacetate)]europate(III) (ATBTA-Eu³⁺) (manufactured by Tokyo Kasei Kogyo Co., Ltd.) and 6 mL of sodium acetate buffer were added to a threaded glass test tube, and a solution of 43 mg of cyanuric chloride (manufactured by Tokyo Kasei Kogyo Co., Ltd.) dissolved in 2.5 mL of acetone was added thereto. After reacting for 1 hour at room temperature, the reaction solution was added to 100 mL of acetone, and the precipitated solid: DTBTA-Eu³⁺ was recovered by centrifugation. Thereafter, it was washed twice with 50 mL of acetone, dried, and dissolved in 100 mL of sodium carbonate buffer to obtain a DTBTA-Eu³⁺ solution.

100 g of the slurry containing the cellulose particles and 100 mL of the prepared DTBTA-Eu³⁺ solution were added to an eggplant-shaped glass flask, a glass reflux tube was attached thereto, and the mixture was stirred with a magnetic stirrer for 3 hours at 60° C. while cooling by refluxing tap water. Thereafter, using a centrifuge, decantation, dilution with deionized water, and washing were repeated several times, followed by a dispersion treatment using a high-pressure homogenizer to obtain 100 g of a fluorescent cellulose particle dispersion.

The obtained fluorescent cellulose particles were placed in an eggplant-shaped glass flask, 200 g of a 4% by mass sodium hydroxide aqueous solution was added as a dispersion medium, 12 g of cyanuric chloride (manufactured by Tokyo Kasei Kogyo Co., Ltd.) was added, a glass reflux tube was attached thereto, and the mixture was stirred with a magnetic stirrer at 60° C. for 3 hours while cooling by refluxing tap water. Thereafter, using a centrifuge, decantation, dilution with deionized water, and washing were performed three times. Thereafter, a dispersion treatment was performed using a high-pressure homogenizer to obtain 100 g of modified fluorescent cellulose particles in the form of a slurry. The average particle size of the obtained fluorescent cellulose particles was measured and found to be 281 nm.

### Example 2

Fluorescent cellulose particles were produced in the same manner as in Example 1, except that the amount of cyanuric chloride used to modify the particles was changed so that the content was as shown in Table 1 below.

### Comparative Example 1

Particle staining was performed in the same manner as in Example 1, and fluorescent cellulose particles without cyanuric chloride modification were produced.

### Comparative Example 2

Fluorescent cellulose particles were produced in the same manner as in Example 1, except that the amount of cyanuric chloride used to modify the particles was changed so that the content was as shown in Table 1 below.

### [Measurement of Fluorescence Intensity of Fluorescent Cellulose Particle Dispersion]

The obtained slurry of the fluorescent cellulose particles was diluted with distilled water so that the cellulose particles were 0.002% by mass to prepare a sample for fluorescence intensity measurement. The sample was placed in a 1 cm square quartz cell, and measured using a spectrofluorophotometer (FP-8300/manufactured by JASCO Corporation) at excitation and fluorescence wavelengths matched to the fluorescent substance.

### [Deployability Test on Test Strip Prepared Using Fluorescent Cellulose Particles]

A test strip was prepared using the obtained fluorescent cellulose particles, and the deployability of the particles in the deployment membrane was evaluated.

The preparation of the test strip will be described below.

20 μL of the dispersion of the fluorescent cellulose particles (Examples 1 and 2, Comparative Examples 1 and 2) having a concentration of 5 mg/ml (dispersion medium: distilled water) and 500 μL of distilled water were added to a microtube, stirred gently, and thereafter, the mixture was centrifuged at 20,000×g for 20 minutes to remove the supernatant. 526 μL of a storage buffer (50 mM borate buffer (pH 10.0), 10% trehalose) was added to disperse the particles to obtain a fluorescent cellulose particle dispersion (0.038%).

424 μL of the dispersion of fluorescent cellulose particles was uniformly applied to a polyester conjugate pad (6613, manufactured by Ahlstrom) (10×160 mm). This was dried in a dryer at 37° C. for 30 minutes to produce a conjugate pad containing the fluorescent cellulose particles.

A sample pad (Microline CBSP097, manufactured by Asahi Kasei Corporation), the conjugate pad, a nitrocellulose membrane without antibody immobilization, and an absorbent pad (Type A/B Extra Thick Glass Fiber 8×10 In, manufactured by PALL) were combined in this order on a backing sheet (product name AR9020, manufactured by Adhesives Research), and this was cut into 4 mm wide and 60 mm long strips to obtain a test strip. Note that each component member was pasted with the ends thereof overlapped with the adjacent member by approximately 2 mm.

80 mL of an immunochromatography deployment solution was dropped onto the sample pad of the prepared test strip, and after 15 minutes, the test strip was evaluated coloration 4 mm upstream of membrane and coloration on the absorbent pad as shown in FIG. 1, using UV light (wavelength: 375 nm). Strong coloration of the absorbent pad means that more particles have flowed to the absorbent pad, i.e., suitable deployability was achieved. In each of 4 mm upstream of membrane, and the absorbent pad, the case in which no coloration was observed was evaluated as (−), the case in which coloration was observed was evaluated as (+), and the case in which strong coloration was observed was evaluated as (++). The results are shown in Table 1 below.

TABLE 1

| | | | | | | Deployability | |
|---|---|---|---|---|---|---|---|
| | | | | | | | Coloration |
| | Fluorescent | fluorescent | Heterocyclic | Average | Dispersion | Coloration | of |
| | dye | dye | compound | particle | fluorescence | upstream of | absorbent |
| | compound | content | content | size | intensity | memembrane | pad |
| | name | % | % | nm | a.u | — | — |
| Ex 1 | ATBTA- | 12 | 6 | 281 | 4590 | − | ++ |
| | Eu³⁺ | | | | | | |
| Ex 2 | ATBTA- | 12 | 3.4 | 280 | 4590 | − | ++ |
| | Eu³⁺ | | | | | | |
| Comp | ATBTA- | 12 | 0 | 272 | 4590 | ++ | − |
| Ex 1 | Eu³⁺ | | | | | | |
| Comp | ATBTA- | 12 | 1.4 | 277 | 4590 | + | + |
| Ex 2 | Eu³⁺ | | | | | | |

From the results shown in Table 1, for the fluorescent cellulose particles of each of Examples 1 and 2, no coloration was observed upstream of the membrane and the coloration of the absorbent pad was strong, confirming suitable deployability. Conversely, in Comparative Example 1, it became poor deployment because fluorescent cellulose particles were clogged upstream of membrane and no farther deployment could be achieved, and there was substantially no coloration of the absorbent pad. In Comparative Example 2, though coloration of the absorbent pad could be confirmed, it was weak as compared to Examples 1 and 2, and since coloration was also confirmed upstream of the membrane, sufficient deployability for immunochromatography could not be obtained.

### Examples 3 and 4

Fluorescent cellulose particles were produced in the same manner as in Example 1, except 115 that the amount of fluorescent dye compound added and the amount of cyanuric chloride used to modify the particles were changed so that the contents thereof were as shown in Table 2 below.

### Examples 5 and 6

Fluorescent cellulose particles were produced in the same manner as in Example 1, except that the fluorescent dye compound was changed to 5-(4,6 dichlorotriazinyl)aminofluorescein (DTAF) (manufactured by Sigma-Aldrich), and the amount of fluorescent dye compound added and the amount of cyanuric chloride used to modify the particles were adjusted so that the contents thereof were as shown in Table 2 below.

### Comparative Example 3

Fluorescent cellulose particles were produced in the same manner as in Example 1, except that the amount of cyanuric chloride used to modify the particles was changed so that the content was as shown in Table 2 below.

### Comparative Examples 4 to 7

Fluorescent cellulose particles were produced in the same manner as in Example 1, except that the amount of fluorescent dye compound added and the amount of cyanuric chloride used to modify the particles were changed so that the contents thereof were as shown in Table 2 below.

### [Measurement of Fluorescence Intensity of Fluorescent Cellulose Particle Dispersion]

The obtained slurry of the fluorescent cellulose particles was diluted with distilled water so that the fluorescent cellulose particles were 0.002% by mass to prepare a sample for fluorescence intensity measurement. The sample was placed in a 1 cm square quartz cell, and measured using a spectrofluorophotometer (FP-8300/manufactured by JASCO Corporation) at excitation and fluorescence wavelengths matched to the fluorescent substance.

### [Color Development Intensity Test Using Immunochromatography Kit Prepared Using Fluorescent Cellulose Particles]

An immunochromatography kit was prepared using the obtained fluorescent cellulose particles, and the color development intensity was evaluated.

The preparation of the immunochromatography kit will be described below.

20 μL of the dispersion of the fluorescent cellulose particles (Examples 1 to 6, Comparative Examples 1 to 7) at a concentration of 5 mg/ml (dispersion medium: distilled water) and 180 μL of a 10 mM phosphate buffer (pH 7.0) were added to a 5 mL tube, and gently stirred. 10 μL (5.8 mg/mL) of anti-hCG antibody (Anti-hCG clone codes/5008, manufactured by Medix Biochemica) was added to the 5 mL tube, and incubated at 37° C. for 2 hours so that the anti-hCG antibody was adsorbed onto the fluorescent cellulose particles.

After incubation, a blocking buffer (100 mM boric acid (pH 8.5), 1% by weight casein) was added to the 5 mL tube, and blocking was performed by incubating at 37° C. for 1 hour.

After blocking, the 5 mL tube was centrifuged at 20,000×g for 15 minutes to remove the supernatant. Next, a washing solution (50 mM borate buffer (pH 10.0)) was added thereto to disperse the particles. After dispersion, the mixture was centrifuged at 20,000×g for 15 minutes to remove the supernatant. A storage buffer (50 mM borate buffer (pH 10.0), 10% trehalose, 4% histidine, 0.4% casein) was added such that the particle weight was 0.038% to disperse the particles, whereby a dispersion of fluorescent cellulose particles/biomolecule composite particles was obtained.

424 μL of the composite particle dispersion was uniformly applied to a polyester conjugate pad (6613, manufactured by Ahlstrom) (10×160 mm). This was dried in a dryer at 37° C. for 30 minutes to prepare a conjugate pad containing the composite particles.

The method for producing an antibody-immobilized membrane will be described below.

A solution ((50 mM KH₂PO₄, pH 7.0)+5% sucrose) containing 1 mg/mL of an anti-hCG antibody (alpha subunit of FSH (LH), clone code/6601, manufactured by Medix Biochemica) was applied at a coating amount of 0.75 μL/cm near the center (approximately 12 mm from the edge) of the membrane (length 25 mm, product name: Hi-Flow Plus 120 membrane, manufactured by MILLIPORE) as a test line having a width of approximately 1 mm.

Next, as a control line having a width of about 1 mm, a solution ((50 mM KH₂PO₄, pH 7.0) sugar free) containing 1 mg/mL of anti-mouse IgG antibody (Anti Mouse IgG, manufactured by Dako) was applied at a coating amount of 0.75 μL/cm, and dried at 50° C. for 30 minutes. Note that the interval between the test line and the control line was 6 mm. Next, as a blocking treatment, the entire membrane was immersed in a blocking buffer (composition: 100 mM boric acid (pH 8.5), 1% by weight casein) at room temperature for 30 minutes.

The membrane was transferred to a membrane washing/stabilizing buffer (composition: 10 mM KH₂PO₄ (pH 7.5), 1% by weight sucrose, 0.1% sodium cholate) and allowed to stand at room temperature for 30 minutes or longer. The membrane was removed, placed on a paper towel, and dried overnight at room temperature to produce an antibody-immobilized membrane.

A sample pad (Microline CBSP097, manufactured by Asahi Kasei Corporation), the conjugate pad, the antibody-immobilized membrane, and an absorbent pad (Type A/B Extra Thick Glass Fiber 8×10 In, manufactured by PALL) were combined in this order on backing sheet (product name AR9020, manufactured by Adhesives Research), and this was cut into strips having a width of 5 mm and a length of 60 mm to obtain a test strip.

Note that each component member was pasted with the ends overlapped with the adjacent member by approximately 2 mm. 80 μL of recombinant hCG (manufactured by Rohto Pharmaceutical Co., Ltd.) at the detection limit concentration (LOD) was dropped onto the sample pad of the prepared test strip, and after 15 minutes, the color development intensity of the test line was evaluated using a fluorescent immunochromatography reader “DxCELL series HRDR-300” manufactured by Cellmic, LLC. Furthermore, 80 μL of a sample containing no antigen was dropped, and the color development intensity of the test line was evaluated in the same manner. The color development observed in samples which did not contain antigens is not color development formed by the correct antibody-antigen reaction and is therefore non-specific color development (noise). The ratio between the non-specific color development and the test line at the detection limit concentration was calculated as the S/N ratio. The S/N ratio is the ratio of signal to noise, and a value greater than 1 indicates that signal is more distinguishable from noise. Specifically, it means that the antigen at the detection limit concentration can be detected. This time, it was determined that the antigen at the detection limit concentration was detectable when the S/N ratio was 2 or more, and undetectable when the S/N ratio was less than 2.

Furthermore, as shown in FIG. 1, the deployed test strip was evaluated coloration 4 mm upstream of membrane, and coloration on the absorbent pad, the case in which no coloration was observed was evaluated as (−), the case in which coloration was observed was evaluated as (+), and the case in which strong coloration was observed was evaluated as (++). The results are shown in Table 2 below.

As shown in Table 2 below, the fluorescent cellulose particles of each of Examples 1 to 6 showed no coloration upstream of membrane and exhibited satisfied deployability and S/N ratio. Conversely, in Comparative Example 1, clogging occurred upstream of membrane, resulting in poor deployment, and the line could not be detected. In Comparative Example 2, some clogging occurred upstream of the membrane, and it was not possible to detect the same antigen concentration as in the other Examples. In Comparative Example 3, though no coloration occurred upstream of the membrane, the coloration of the absorbent pad was weaker than in the Examples, since the test line intensity was weak, it was not possible to detect the same antigen concentration as in the other Examples. Therefore, it didn't have sufficient deployability for immunochromatography. In Comparative Example 4, since clogging occurred upstream of the membrane, the coloration of the absorbent pad was weaker than in the other Examples and the test line intensity was also weak, the S/N ratio was small. Thus, it didn't have sufficient deployability for immunochromatography. In Comparative Example 5, though the line intensity value was high, it resulted in a low S/N ratio because the coloration was strong from the upstream side of the membrane to the entire membrane and the color development was strong even in non-specific cases. In Comparative Example 6, the same antigen concentration as the other Examples could not be detected because the brightness of the particles was weak. In Comparative Example 7, though no coloration was observed upstream of the membrane, concentration quenching occurred and the brightness of the particles became weaker, resulting in lower sensitivity, and the same antigen concentration as in the other Examples could not be detected.

TABLE 2

| | | | | | | Immuno performance | | | | |
|---|---|---|---|---|---|---|---|---|---|---|
| | | | | | | | | | Coloration | |
| | Fluorescent | fluorescent | Heterocyclic | Average | Dispersion | Coloration | Line | Line | of | |
| | dye | dye | compound | particle | fluorescence | upstream of | intensity | intensity | absorbent | S/N ratio |
| | compound | content | content | size | intensity | membrane | (non-specific) | (LOD) | pad | (LOD/non- |
| | name | % | % | nm | a.u | — | a.u. | a.u. | — | specific) |
| Ex 1 | ATBTA- | 12 | 6 | 281 | 4590 | − | 1 | 4.5 | ++ | 4.5 |
| | Eu³⁺ | | | | | | | | | |
| Ex 2 | ATBTA- | 12 | 3.4 | 280 | 4590 | − | 0.5 | 2 | ++ | 4 |
| | Eu³⁺ | | | | | | | | | |
| Ex 3 | ATBTA- | 5 | 45 | 280 | 2138 | − | 0 | 2 | ++ | 2※ |
| | Eu³⁺ | | | | | | | | | |
| Ex 4 | ATBTA- | 35 | 16 | 293 | 7096 | − | 1.25 | 7 | ++ | 5.6 |
| | Eu³⁺ | | | | | | | | | |
| Ex 5 | DTAF | 1 | 34 | 268 | 1496 | − | 1 | 2.5 | ++ | 2.5 |
| Ex 6 | DTAF | 40 | 4 | 307 | 4157 | − | 1.25 | 5 | ++ | 4 |
| Comp | ATBTA- | 12 | 0 | 272 | 4590 | ++ | − | − | − | − |
| Ex 1 | Eu³⁺ | | | | | | | | | |
| Comp | ATBTA- | 12 | 1.4 | 277 | 4590 | + | 0.5 | 0.5 | + | 1 |
| Ex 2 | Eu³⁺ | | | | | | | | | |
| Comp | ATBTA- | 12 | 2.7 | 280 | 4590 | − | 1 | 1 | + | 1 |
| Ex 3 | Eu³⁺ | | | | | | | | | |
| Comp | ATBTA- | 19 | 1 | 275 | 3325 | ++ | 1 | 1.5 | + | 1.5 |
| Ex 4 | Eu³⁺ | | | | | | | | | |
| Comp | ATBTA- | 10 | 53 | 348 | 3552 | + | 3.5 | 4.75 | − | 1.36 |
| Ex 5 | Eu³⁺ | | | | | | | | | |
| Comp | ATBTA- | 0.7 | 10 | 265 | 640 | − | 0.5 | 0.5 | + | 1 |
| Ex 6 | Eu³⁺ | | | | | | | | | |
| Comp | ATBTA- | 46 | 8 | 314 | 1081 | − | 1 | 1.5 | + | 1.5 |
| Ex 7 | Eu³⁺ | | | | | | | | | |

※Since the denominator is 0 and calculation cannot be performed, the difference between LOD and non-specific was used

### INDUSTRIAL APPLICABILITY

Since the fluorescent cellulose particles of the present invention and the immunochromatography kit using the same can detect substances to be detected contained in a biological sample with high sensitivity, they can suitably be used in immunoassays in clinical tests and the like.

1: Fluorescent cellulose particles, comprising cellulose particles, a fluorescent dye compound, and a heterocyclic compound represented by general formula (1) below: 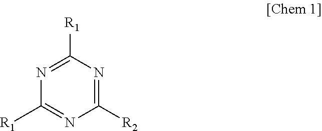
where R₁ is a functional group having affinity with a biological material, and R₂ is an ether bond to the cellulose particles, wherein per gram of the fluorescent cellulose particles, a content of the cellulose particles is 30% by mass or more and 90% by mass or less, a content of the fluorescent dye compound is 1% by mass or more and 40% by mass or less, and a content of the heterocyclic compound is 3% by mass or more and 50% by mass or less.

2: The fluorescent cellulose particles according to claim 1, wherein R₁ of the heterocyclic compound is Cl and/or OH.

3: The fluorescent cellulose particles according to claim 1, wherein an average particle diameter of the fluorescent cellulose particles is 9 nm or more and 500 nm or less.

4: The fluorescent cellulose particles according to claim 1, wherein the fluorescent dye compound is bonded to an OH group of the cellulose particles, and the heterocyclic compound is bonded to an OH group of the cellulose particles.

5: The fluorescent cellulose particles according to claim 1, wherein the fluorescent dye compound is a europium complex.

6: The fluorescent cellulose particles according to claim 1, wherein the biological material is supported via physical adsorption.

7: The fluorescent cellulose particles according to claim 6, wherein the biological material is a protein, a peptide, or a nucleic acid.

8: The fluorescent cellulose particles according to claim 7, wherein the protein is an antigen or an antibody.

9: A diagnostic agent comprising the fluorescent cellulose particles according to claim 1.

10: An immunochromatography kit comprising the fluorescent cellulose particles according to claim 1.
